(19) United States
(12) Patent Application Publication
(10) Pub. No.: US 2019/0159666 A1
(43) Pub. Date: May 30, 2019

(54) SURGICAL ILLUMINATION SYSTEM

(71) Applicant: Acclarent, Inc., Irvine, CA (US)

(72) Inventor: George L. Matlock, Pleasanton, CA (US)

(21) Appl. No.: 15/822,424

(22) Filed: Nov. 27, 2017

(51) Int. Cl. A61B 1/233; A61B 1/06; A61B 1/00; A61M 25/09; G02B 27/14; G02B 26/02

(52) U.S. Cl. CPC A61B 1/233; A61B 1/0661; A61B 1/00117; A61M 29/02; G02B 27/14; G02B 26/02; A61M 25/09

(57) ABSTRACT

A guide system includes a first instrument, a second instrument, a light source, and a light splitter. A distal portion of each instrument is configured to fit within a nasal cavity of a patient. A distal portion of each instrument is further configured to project light. The light source is operable to simultaneously project light to the first and second instruments through at least one light cable such that the at least one light cable is configured to optically couple the light source to the first and second instruments. The light splitter is in optical communication with the light source and the first and second instruments such that the light splitter is interposed between the light source and the first and second instruments. The light splitter is configured to selectively bifurcate the light projected from the light source to the first and second instruments

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and guide catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pub. No. 2011/0004057, entitled “Systems and Methods for Transnasal Dilation of Passageways in the Ear, Nose or Throat,” published Jan. 6, 2011, the disclosure of which is incorporated by reference herein. An example of such a system is the Relieva® Spin Balloon Sinuplasty™ System by Acclarent, Inc. of Irvine, Calif.

A variable direction view endoscope may be used with such a system to provide visualization within the anatomical passageway (e.g., the ear, nose, throat, paranasal sinuses, etc.) to position the balloon at desired locations. A variable direction view endoscope may enable viewing along a variety of transverse viewing angles without having to flex the shaft of the endoscope within the anatomical passageway. Such an endoscope that may be provided in accordance with the teachings of U.S. Pub. No. 2010/0030031, entitled “Swing Prism Endoscope,” published Feb. 4, 2010, the disclosure of which is incorporated by reference herein.

While a variable direction view endoscope may be used to provide visualization within the anatomical passageway, it may also be desirable to provide additional visual confirmation of the proper positioning of the balloon before inflating the balloon. This may be done using an illuminating guidewire. Such a guidewire may be positioned within the target area and then illuminated, with light projecting from the distal end of the guidewire. This light may illuminate the adjacent tissue (e.g., hypodermis, subdermis, etc.) and thus be visible to the naked eye from outside the patient through transcutaneous illumination. For instance, when the distal end is positioned in the maxillary sinus, the light may be visible through the patient's cheek. Using such external visualization to confirm the position of the guidewire, the balloon may then be advanced distally along the guidewire into position at the dilation site. Such an illuminating guidewire may be provided in accordance with the teachings of U.S. Pat. No. 9,155,492, entitled “Sinus Illumination Lightwire Device,” issued Oct. 13, 2015, the disclosure of which is incorporated by reference herein. An example of such an illuminating guidewire is the Relieva Luma Sentry™ Sinus Illumination System by Acclarent, Inc. of Irvine, Calif.

It may be desirable to provide easily controlled placement of a balloon in dilation procedures, including procedures that will be performed only by a single operator. While several systems and methods have been made and used to inflate an inflatable member such as a dilation balloon, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a side elevational view of an exemplary dilation catheter system;

FIG. 2A depicts a side elevational view of an exemplary illuminating guidewire of the dilation catheter system of FIG. 1;

FIG. 2B depicts a side elevational view of an exemplary guide catheter of the dilation catheter system of FIG. 1;

FIG. 2C depicts a side elevational view of an exemplary dilation catheter of the dilation catheter system of FIG. 1;

FIG. 3 depicts a detailed side elevational view of the illuminating guidewire of FIG. 2A;

FIG. 4 depicts a detailed side cross-sectional view of the illuminating guidewire of FIG. 2A;

FIG. 5 depicts a perspective view of an exemplary endoscope suitable for use with the dilation catheter system of FIG. 1;

FIG. 6 depicts a side elevational view of the distal end of the endoscope of FIG. 5, showing an exemplary range of viewing angles;

FIG. 7A depicts a front view of the guide catheter of FIG. 2B positioned adjacent an ostium of the maxillary sinus;

FIG. 7B depicts a front view of the guide catheter of FIG. 2B positioned adjacent an ostium of the maxillary sinus, with the dilation catheter of FIG. 2C and the illuminating guidewire of FIG. 2A positioned in the guide catheter and a distal portion of the guidewire positioned in the maxillary sinus;

FIG. 7C depicts a front view of the guide catheter of FIG. 2B positioned adjacent an ostium of the maxillary sinus, with the illuminating guidewire of FIG. 2A translated further distally relative to the guide catheter and into the maxillary sinus;

FIG. 7D depicts a front view of the guide catheter of FIG. 2B positioned adjacent an ostium of the maxillary sinus, with the dilation catheter of FIG. 2C translated distally relative to the guide catheter along the illuminating guidewire of FIG. 2A so as to position a balloon of the dilation catheter within the ostium;

FIG. 7E depicts a front view of an ostium of the maxillary sinus, with the ostium having been enlarged by inflation of the balloon of FIG. 7D;

FIG. 8 depicts a schematic view of an exemplary illuminating system with the illuminating guidewire of FIG. 2A and the endoscope of FIG. 5 connected to a single light source, the light source including a pair of cables and a connector with an integral light splitter; and

FIG. 9 depicts a schematic view of another exemplary illuminating system with the illuminating guidewire of FIG. 2A and the endoscope of FIG. 5 connected to a single light source, the light source including two cables and a light source with an integral light splitter.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. For example, while various. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Overview of Exemplary Dilation Catheter System

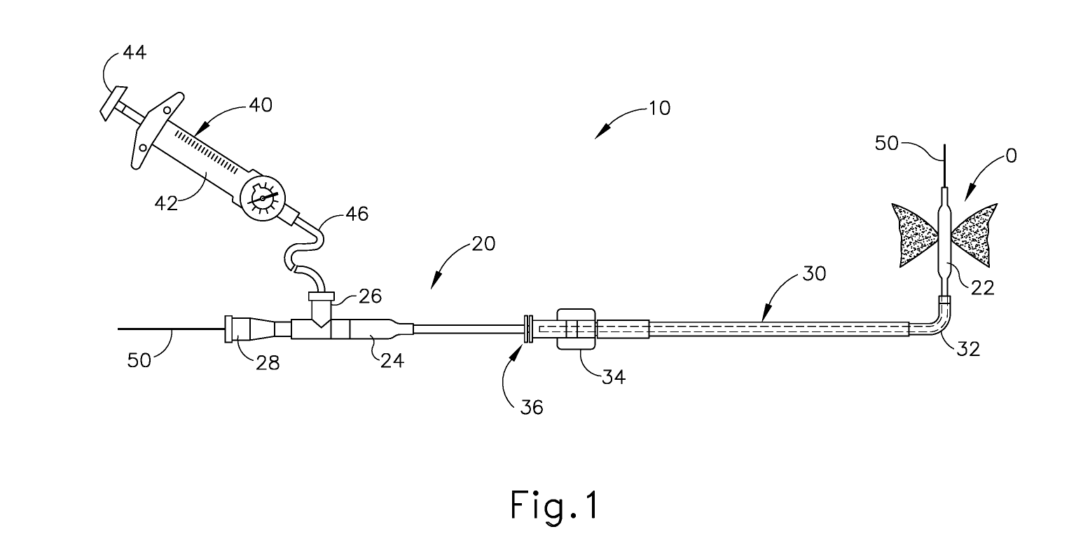

FIG. 1 shows an exemplary dilation catheter system (10) that may be used to dilate the ostium of a paranasal sinus; or to dilate some other anatomical passageway (e.g., within the ear, nose, or throat, etc.). Dilation catheter system (10) of this example comprises a dilation catheter (20), a guide catheter (30), an inflator (40), and a guidewire (50). By way of example only, dilation catheter system (10) may be configured in accordance with at least some of the teachings of U.S. Patent Pub. No. 2011/0004057, the disclosure of which is incorporated by reference herein. In some versions, at least part of dilation catheter system (10) is configured similar to the Relieva® Spin Balloon Sinuplasty™ System by Acclarent, Inc. of Irvine, Calif.

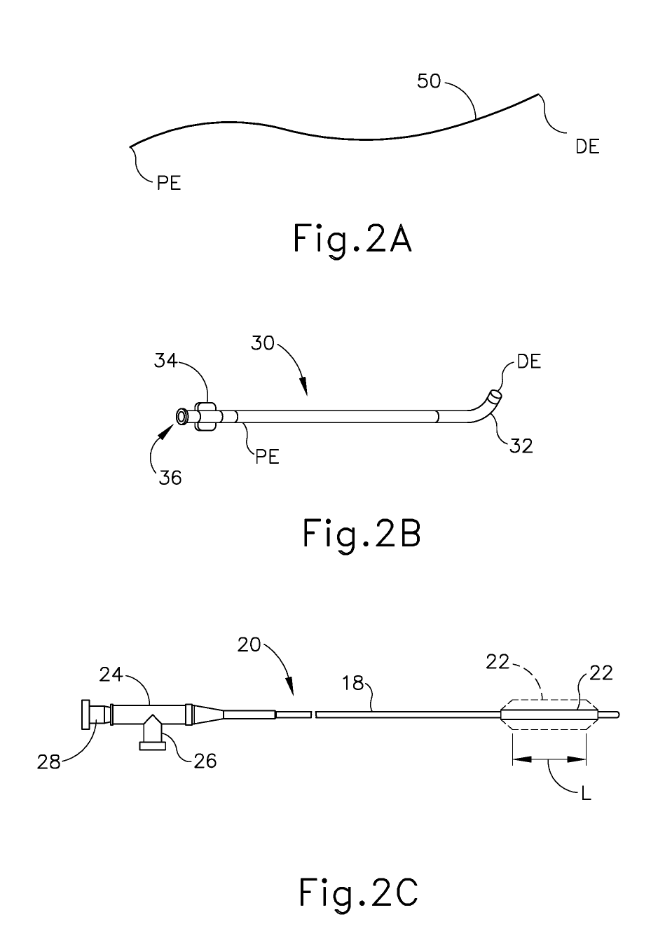

As best seen in FIG. 2C, the distal end (DE) of dilation catheter (20) includes an inflatable dilator (22). The proximal end (PE) of dilation catheter (20) includes a grip (24), which has a lateral port (26) and an open proximal end (28). A hollow-elongate shaft (18) extends distally from grip. Dilation catheter (20) includes a first lumen (not shown) formed within shaft (18) that provides fluid communication between lateral port (26) and the interior of dilator (22). Dilator catheter (20) also includes a second lumen (not shown) formed within shaft (18) that extends from open proximal end (28) to an open distal end that is distal to dilator (22). This second lumen is configured to slidably receive guidewire (50). The first and second lumens of dilator catheter (20) are fluidly isolated from each other. Thus, dilator (22) may be selectively inflated and deflated by communicating fluid along the first lumen via lateral port (26) while guidewire (50) is positioned within the second lumen. In some versions, dilator catheter (20) is configured similar to the Relieva Ultirra™ Sinus Balloon Catheter by Acclarent, Inc. of Irvine, Calif. In some other versions, dilator catheter (20) is configured similar to the Relieva Solo Pro™ Sinus Balloon Catheter by Acclarent, Inc. of Irvine, Calif. Other suitable forms that dilator catheter (20) may take will be apparent to those of ordinary skill in the art in view of the teachings herein.

As best seen in FIG. 2B, guide catheter (30) of the present example includes a bent distal portion (32) at its distal end (DE) and a grip (34) at its proximal end (PE). Grip (34) has an open proximal end (36). Guide catheter (30) defines a lumen that is configured to slidably receive dilation catheter (20), such that guide catheter (30) may guide dilator (22) out through bent distal end (32). In some versions, guide catheter (30) is configured similar to the Relieva Flex™ Sinus Guide Catheter by Acclarent, Inc. of Irvine, Calif. Other suitable forms that guide catheter (30) may take will be apparent to those of ordinary skill in the art in view of the teachings herein.

Referring back to FIG. 1, inflator (40) of the present example comprises a barrel (42) that is configured to hold fluid and a plunger (44) that is configured to reciprocate relative to barrel (42) to selectively discharge fluid from (or draw fluid into) barrel (42). Barrel (42) is fluidly coupled with lateral port (26) via a flexible tube (46). Thus, inflator (40) is operable to add fluid to dilator (22) or withdraw fluid from dilator (22) by translating plunger (44) relative to barrel (42). In the present example, the fluid communicated by inflator (40) comprises saline, though it should be understood that any other suitable fluid may be used. There are various ways in which inflator (40) may be filled with fluid (e.g., saline, etc.). By way of example only, before flexible tube (46) is coupled with lateral port (26), the distal end of flexible tube (46) may be placed in a reservoir containing the fluid. Plunger (44) may then be retracted from a distal position to a proximal position to draw the fluid into barrel (42). Inflator (40) may then be held in an upright position, with the distal end of barrel (42) pointing upwardly, and plunger (44) may then be advanced to an intermediate or slightly distal position to purge any air from barrel (42). The distal end of flexible tube (46) may then be coupled with lateral port (26). In some versions, inflator (40) is constructed and operable in accordance with at least some of the teachings of U.S. Pub. No. 2014/0074141, entitled “Inflator for Dilation of Anatomical Passageway,” published Mar. 13, 2014, the disclosure of which is incorporated by reference herein.

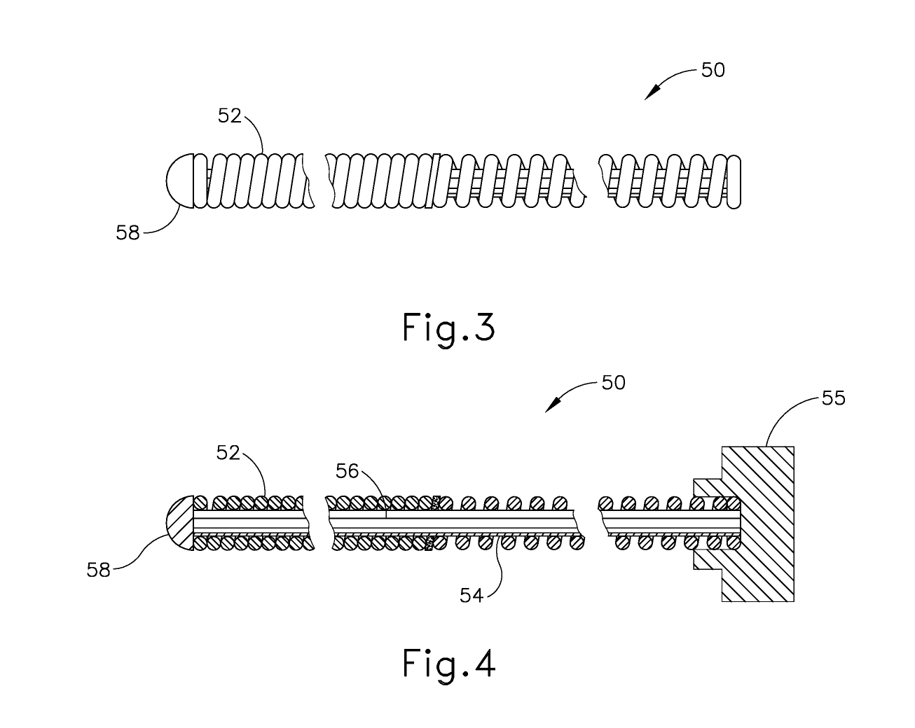

As shown in FIGS. 2A, 3, and 4, guidewire (50) of the present example comprises a coil (52) positioned about a core wire (54). An illumination fiber (56) extends along the interior of core wire (54) and terminates in an atraumatic lens (58). A connector (55) at the proximal end of guidewire (50) enables optical coupling between illumination fiber (56) and a light source (not shown). Illumination fiber (56) may comprise one or more optical fibers. Lens (58) is configured to project light when illumination fiber (56) is illuminated by the light source, such that illumination fiber (56) transmits light from the light source to the lens (58). In some versions, the distal end of guidewire (50) is more flexible than the proximal end of guidewire (50). Guidewire (50) has a length enabling the distal end of guidewire (50) to be positioned distal to dilator (22) while the proximal end of guidewire (50) is positioned proximal to grip (24). Guidewire (50) may include indicia along at least part of its length (e.g., the proximal portion) to provide the operator with visual feedback indicating the depth of insertion of guidewire (50) relative to dilation catheter (20). By way of example only, guidewire (50) may be configured in accordance with at least some of the teachings of U.S. Pat. No. 9,155,492, the disclosure of which is incorporated by reference herein. In some versions, guidewire (50) is configured similar to the Relieva Luma Sentry™ Sinus Illumination System by Acclarent, Inc. of Irvine, Calif. Other suitable forms that guidewire (50) may take will be apparent to those of ordinary skill in the art in view of the teachings herein.

II. Overview of Exemplary Endoscope

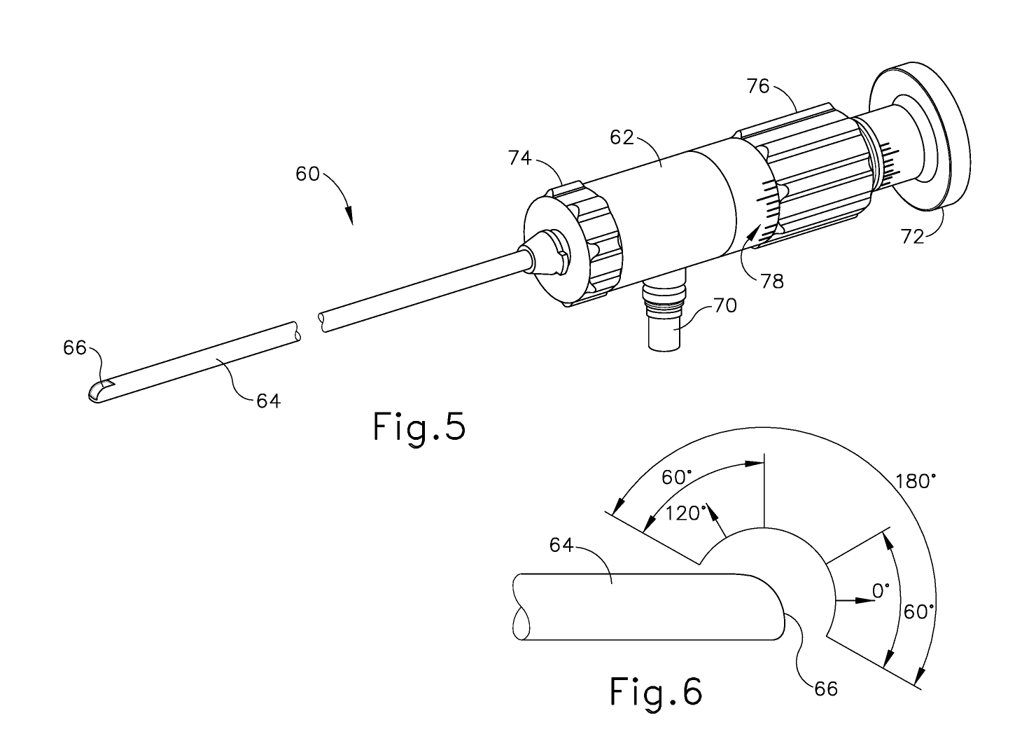

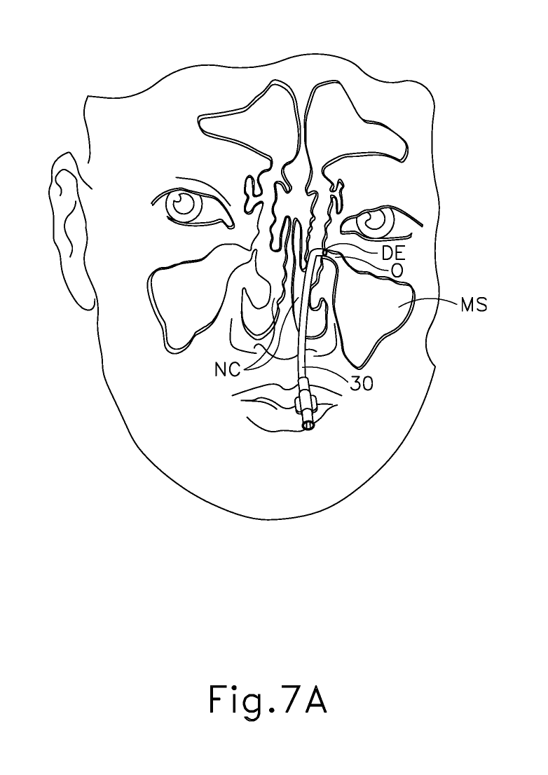

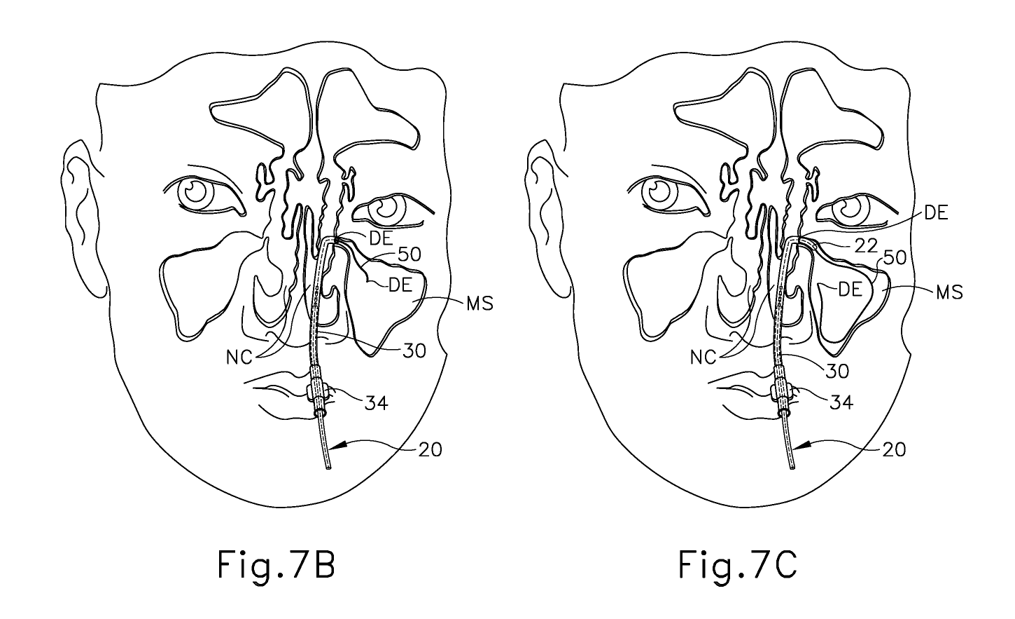

As noted above, an endoscope (60) may be used to provide visualization within an anatomical passageway (e.g., within the nasal cavity, etc.) during a process of using dilation catheter system (10). As shown in FIGS. 4-5, endoscope of the present example comprises a body (62) and a rigid shaft (64) extending distally from body (62). The distal end of shaft (64) includes a curved transparent window (66). A plurality of rod lenses and light transmitting fibers may extend along the length of shaft (64). A lens is positioned at the distal end of the rod lenses and a swing prism is positioned between the lens and window (66). The swing prism is pivotable about an axis that is transverse to the longitudinal axis of shaft (64). The swing prism defines a line of sight that pivots with the swing prism. The line of sight defines a viewing angle relative to the longitudinal axis of shaft (64). This line of sight may pivot from approximately 0 degrees to approximately 120 degrees, from approximately 10 degrees to approximately 90 degrees, or within any other suitable range. The swing prism and window (66) also provide a field of view spanning approximately 60 degrees (with the line of sight centered in the field of view). Thus, the field of view enables a viewing range spanning approximately 180 degrees, approximately 140 degrees, or any other range, based on the pivot range of the swing prism. Of course, all of these values are mere examples.

Body (62) of the present example includes a light post (70), an eyepiece (72), a rotation dial (74), and a pivot dial (76). Light post (70) is in communication with the light transmitting fibers in shaft (64) and is configured to couple with a source of light, to thereby illuminate the site in the patient distal to window (66). Eyepiece (72) is configured to provide visualization of the view captured through window (66) via the optics of endoscope (60). It should be understood that a visualization system (e.g., camera and display screen, etc.) may be coupled with eyepiece (72) to provide visualization of the view captured through window (66) via the optics of endoscope (60). Rotation dial (74) is configured to rotate shaft (64) relative to body (62) about the longitudinal axis of shaft (64). It should be understood that such rotation may be carried out even while the swing prism is pivoted such that the line of sight is non-parallel with the longitudinal axis of shaft (64). Pivot dial (76) is coupled with the swing prism and is thereby operable to pivot the swing prism about the transverse pivot axis. Indicia (78) on body (62) provide visual feedback indicating the viewing angle. Various suitable components and arrangements that may be used to couple rotation dial (74) with the swing prism will be apparent to those of ordinary skill in the art in view of the teachings herein. By way of example only, endoscope (60) may be configured in accordance with at least some of the teachings of U.S. Pub. No. 2010/0030031, the disclosure of which is incorporated by reference herein. Other suitable forms that endoscope (60) may take will be apparent to those of ordinary skill in the art in view of the teachings herein

III. Exemplary Method for Dilating the Ostium of a Maxillary Sinus

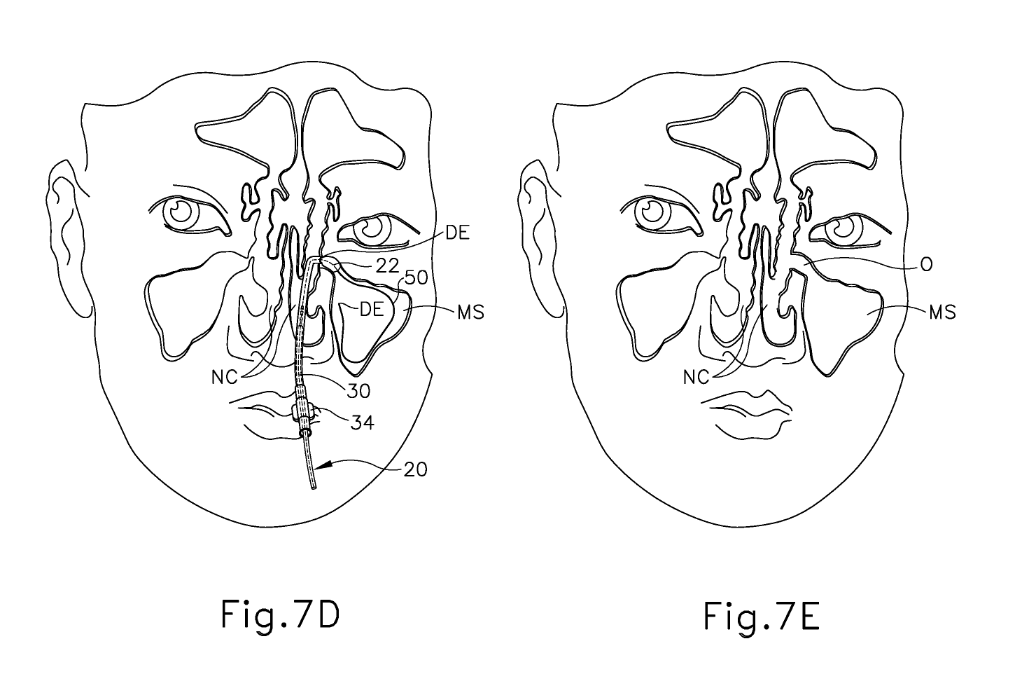

FIGS. 7A-7E show an exemplary method for using dilation catheter system (10) discussed above to dilate a sinus ostium (O) of a maxillary sinus (MS) of a patient. While the present example is being provided in the context of dilating a sinus ostium (O) of a maxillary sinus (MS), it should be understood that dilation catheter system (10) may be used in various other procedures. By way of example only, dilation catheter system (10) and variations thereof may be used to dilate a Eustachian tube, a larynx, a choana, a sphenoid sinus ostium, one or more openings associated with one or more ethmoid sinus air cells, the frontal recess, and/or other passageways associated with paranasal sinuses. Other suitable ways in which dilation catheter system (10) may be used will be apparent to those of ordinary skill in the art in view of the teachings herein.

In the procedure of the present example, guide catheter (30) may be inserted transnasally and advanced through the nasal cavity (NC) to a position within or near the targeted anatomical passageway to be dilated, the sinus ostium (O), as shown in FIG. 7A. Inflatable dilator (22) and the distal end of guidewire (50) may be positioned within or proximal to bent distal end (32) of guide catheter (30) at this stage. This positioning of guide catheter (30) may be verified endoscopically with an endoscope such as endoscope (60) described above and/or by direct visualization, radiography, and/or by any other suitable method. After guide catheter (30) has been positioned, the operator may advance guidewire (50) distally through guide catheter (30) such that a distal portion of the guidewire (50) passes through the ostium (O) of the maxillary sinus (MS) and into the cavity of the maxillary sinus (MS) as shown in FIGS. 7B and 7C. The operator may illuminate illumination fiber (56) and lens (58), which may provide transcutaneous illumination through the patient's face to enable the operator to visually confirm positioning of the distal end of guidewire (50) in the maxillary sinus (MS) with relative ease.

As shown in FIG. 7C, with guide catheter (30) and guidewire (50) suitably positioned, dilation catheter (20) is advanced along guidewire (50) and through bent distal end (32) of guide catheter (30), with dilator (22) in a non-dilated state until dilator (22) is positioned within the ostium (O) of the maxillary sinus (MS) (or some other targeted anatomical passageway). After dilator (22) has been positioned within the ostium (O), dilator (22) may be inflated, thereby dilating the ostium (O), as shown in FIG. 7D. To inflate dilator (22), plunger (44) may be actuated to push saline from barrel (42) of inflator (40) through dilation catheter (20) into dilator (22). The transfer of fluid expands dilator (22) to an expanded state to open or dilate the ostium (O), such as by remodeling the bone, etc., forming ostium (O). By way of example only, dilator (22) may be inflated to a volume sized to achieve about 10 to about 12 atmospheres. Dilator (22) may be held at this volume for a few seconds to sufficiently open the ostium (O) (or other targeted anatomical passageway). Dilator (22) may then be returned to a non-expanded state by reversing plunger (44) of inflator (40) to bring the saline back to inflator (40). Dilator (22) may be repeatedly inflated and deflated in different ostia and/or other targeted anatomical passageways. Thereafter, dilation catheter (20), guidewire (50), and guide catheter (30) may be removed from the patient as shown in FIG. 7E.

In some instances, it may be desirable to irrigate the sinus and paranasal cavity after dilation catheter (20) has been used to dilate the ostium (O). Such irrigation may be performed to flush out blood, etc. that may be present after the dilation procedure. For example, in some cases, guide catheter (30) may be allowed to remain in place after removal of guidewire (50) and dilation catheter (20) and a lavage fluid, other substance, or one or more other devices (e.g., lavage catheters, balloon catheters, cutting balloons, cutters, chompers, rotating cutters, rotating drills, rotating blades, sequential dilators, tapered dilators, punches, dissectors, burs, non-inflating mechanically expandable members, high frequency mechanical vibrators, dilating stents and radiofrequency ablation devices, microwave ablation devices, laser devices, snares, biopsy tools, scopes, and devices that deliver diagnostic or therapeutic agents) may be passed through guide catheter (30) for further treatment of the condition. By way of example only, irrigation may be carried out in accordance with at least some of the teachings of U.S. Pat. No. 7,630,676, entitled “Methods, Devices and Systems for Treatment and/or Diagnosis of Disorders of the Ear, Nose and Throat,” issued Dec. 8, 2009, the disclosure of which is incorporated by reference herein. An example of an irrigation catheter that may be fed through guide catheter (30) to reach the irrigation site after removal of dilation catheter (20) is the Relieva Vortex® Sinus Irrigation Catheter by Acclarent, Inc. of Irvine, Calif. Another example of an irrigation catheter that may be fed through guide catheter (30) to reach the irrigation site after removal of dilation catheter (20) is the Relieva Ultirra® Sinus Irrigation Catheter by Acclarent, Inc. of Irvine, Calif. Of course, irrigation may be provided in the absence of a dilation procedure; and a dilation procedure may be completed without also including irrigation.

IV. Exemplary Illumination System

As briefly discussed above, in the procedure of the present example both guidewire (50) and endoscope (60) require a source of light to allow each instrument to provide adequate lighting for an operator to visually identify the respective locations of the instruments within a patient. A first light source is provided within the procedure room for purposes of connecting to guidewire (50) and providing light to illuminating fiber (56), while a second light source is also provided in the procedure room to connect with endoscope (60) to provide light to a light pipe or other light guide in shaft (64), to illuminate the field of view at the distal end of shaft (64).

In some instances, it may be beneficial to provide a single light source to supply illumination to both guidewire (50) and endoscope (60). In this instance, the number of pieces of equipment required to be physically present in the procedure room is reduced as the same light source provides both instruments (50, 60) with sufficient lighting for each to perform their respective functions effectively. Furthermore, providing a single light source for the procedure of the present example also serves to minimize the baseline costs associated for an operator to perform the procedure. With the light communicating features of guidewire (50) and endoscope (60) being supplied with light from the same source, a reduction in the necessary equipment to perform the procedure may effectively reduce the overhead costs of including an additional light source for the operator. Additionally, without the need to supply an additional light source for a procedure, the costs associated for an operator and a patient are also effectively reduced for each procedure performed.

This single light source system may further maximize an operator's maneuverability during a procedure as the reduction in the necessary equipment present in the procedure room provides the operator greater space for movement. As described below, a single light source may effectively supply multiple instruments with light through the use of a light splitter that is able to distribute the light transmitted from the light source to the various instruments through one or more conduits or cables. Additionally, in instances where the light communicating features of an instrument are supplied light through the use of an integral light source that is powered by an attached battery, providing a single source of light to supply each instrument in the procedure room with light will eliminate the need to attach a battery to the instrument, thereby decreasing the weight of the instrument. In this instance, the instrument may be easier for an operator to hold and maneuver during the procedure due to the decrease in overall weight of the instrument.

The following description provides various examples of an illuminating system including only one single light source and light splitter, and corresponding surgical instruments, such as a guidewire and endoscope, that are cooperatively configured to connect to the single light source of the illuminating system through the light splitter. Ultimately, the use of the illuminating system may be desirable to reduce the amount of equipment and cables in a procedure room and to minimize costs associated with performing a procedure. It should be understood that the illuminating system described below may be readily combined with any of the various instruments described above and in any of the various surgical procedures described in the various references described herein. Other suitable ways in which the below described illuminating systems may be used will be apparent to those of ordinary skill in the art in view of the teachings herein.

A. Illuminating System Using a Connector with Integral Light Splitter

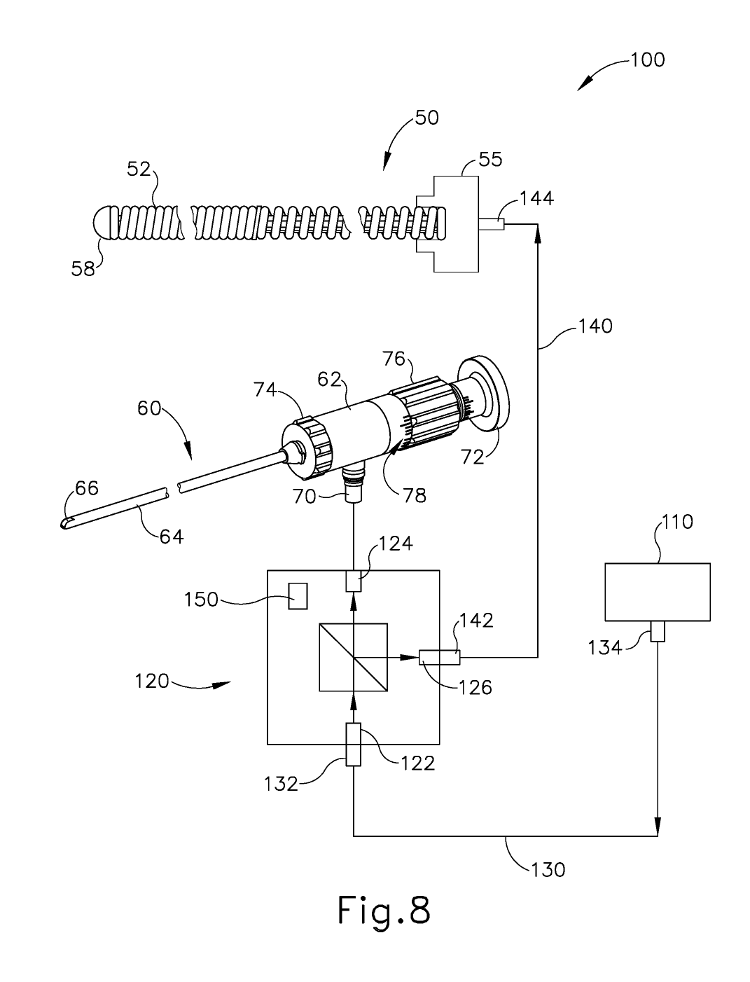

FIG. 8 shows an exemplary illuminating system (100) comprising a light source (110), a light splitter connector (120), and light cables (130, 140). Illuminating system (100) is operable to supply light from light source (110) to multiple instruments connected thereto, through light splitter connector (120) and a series of cables (130, 140), to thereby provide the instruments with sufficient lighting for use in a procedure. Light source (110) may comprise any suitable kind of light source (110) and may include various components, including but not limited to a laser, a beam collimator, focusing optics, etc. Light source (110) may be operable to communicate any suitable kind of light, including but not limited to white/visible light, near-infrared light, infrared light, etc. It should be understood that illuminating system (100) of the present example may be readily combined with guidewire (50) and endoscope (60) described above.

In the present example, as seen in FIG. 8, guidewire (50) and endoscope (60) are included in illuminating system (100) such that illuminating system (100) provides guidewire (50) and endoscope (60) with sufficient lighting to allow an operator to navigate the instruments (50, 60) within a patient. As previously described above, endoscope (60) may be used to provide visualization within an anatomical passageway, such as within the nasal cavity of a patient. While endoscope (60) may be used to provide visualization within the anatomical passageway, it may also be desirable to provide additional visual confirmation of the proper positioning of inflatable dilator or balloon (22) of dilation catheter (20) before inflating dilator (22). As described above, this may be done using illuminating guidewire (50), which may be positioned within the target area and then illuminated, with light projecting from the distal end of guidewire (50) to provide a transillumination effect through the skin of the patient. Although not shown, it should be understood that illuminating system (100) may be connected to other instruments, in addition to or in place of endoscope (60) and guidewire (50), that require a source of illumination.

Light splitter connector (120) of the present example comprises conventional light-splitting components that are operable to split a single beam of light from light source (110) into two beams of light, as will be apparent to those of ordinary skill in the art. By way of example only, light splitter connector (120) may include a pair of triangular glass prisms that are joined together at their hypotenuses. In the present example, light splitter connector (120) is operable to reflect light at an angle of 90° from the axis in which the light was initially received.

In the present example, light splitter connector (120) is directly coupled with light post (70) of endoscope (60) as described below. In some other variations, light splitter connector is integrated into light post (70) of endoscope (60). By way of example only, light splitter connector (120) may comprise a machine fabricated metal assembly that is configured to connect to light source (110), cables (130, 140), endoscope (60), and/or guidewire (50). Alternatively, light splitter connector (120) may be formed of a molded plastic, a potted single outer covering, or other various suitable materials as will be apparent to those of ordinary skill in the art. Light cable (130) comprises a pair of luer connectors (132, 134) positioned at opposite ends of light cable (130). It should be understood that luer connectors (132, 134) are configured and operable to be interchangeable with one another.

As seen in FIG. 8, one luer connector (132) is optically coupled to light splitter connector (120) at a corresponding luer connector (122) of light splitter connector (120) while the other luer connector (134) is coupled to light source (110), such that light source (110) is in optical communication with light splitter connector (120) via light cable (130). Light splitter connector (120) is further coupled to light post (70) of endoscope (60), at another corresponding luer connector (124) of light splitter connector (120), such that light source (110) is in optical communication with endoscope (60) through light splitter connector (120) and light cable (120). In other words, light splitter connector (120) is configured to receive luer connector (132), at corresponding luer connector (122), and light post (70) of endoscope (60) at another luer connector (124). As described above, light post (70) is in optical communication with the light communicating features in shaft (64) of endoscope (60). In this instance, light source (110) is operable to supply the light communicating features of endoscope (60) with light, through cable (130) and light splitter connector (120), such that endoscope (60) is operable to direct the light through transparent window (66) and thereby illuminate the anatomy outside of window (66).

Light splitter connector (120) is further configured to receive a second light cable (140) at another corresponding luer connector (126) of light splitter connector (120). In particular, second cable (140) includes a pair of luer connectors (142, 144) positioned at opposite ends of cable (140). Similar to luer connectors (132, 134) of cable (130), luer connectors (142, 144) of cable (140) are configured and operable to be interchangeable with one another. It should be understood that cable (140) is configured and operable just like cable (130) such that cables (130, 140) are interchangeable. Additionally, it should be understood that corresponding luer connectors (122, 124, 126) of light splitter connector (120) are similarly configured and operable such that corresponding luer connectors (122, 124, 126) are interchangeable. In other words, any corresponding luer connector (122, 124, 126) is configured to receive any luer connector (132, 134, 142, 144) of cable (130, 140) or light post (70) of endoscope (60) therein.

As further seen in FIG. 8, luer connector (142) of cable (140) is coupled to corresponding luer connector (126) of light splitter connector (120), while the opposite luer connector (144) of cable (140) is coupled to connector (55) of guidewire (50). In this instance, light source (110) is in optical communication with endoscope (60) through the connections of cable (130) and light splitter connector (120), and in further communication with guidewire (50) through the connection of cable (140) and light splitter connector (120). Thus, light source (110) is operable to transmit light to guidewire (50) such that illumination fiber (56) receives light and transmits the light to lens (58). Although light splitter connector (120) is shown to include three corresponding luer connectors (122, 124, 126), it should be understood that light splitter connector (120) may include additional corresponding luer connectors (122, 124, 126) such that light splitter connector (120) is configured to connect to more cables and/or instruments.

Light splitter connector (120) of the present example further includes an adjustment gauge (150) that is configured to selectively adjust the intensity of light reflected from light source (110) through light splitter connector (120) and toward guidewire (50) and endoscope (60), respectively. In other words, adjustment gauge (150) is operable to transition the luminous intensity transmitted from light source (110) to each instrument connected within illuminating system (100). In the present example, adjustment gauge (150) has a slotted window (not shown) within light splitter connector (120), the position of which is selectively adjustable to control the luminance transmitted to the respective instruments connected to light splitter connector (120). In other examples, adjustment gauge (150) may comprise a series of apertures or holes (not shown) that have varying diameters. In this instance, the intensity of light transmitted to guidewire (50) or endoscope (60) is determined by the particular diameter of the hole selected by an operator.

Adjustment gauge (150) may further include a rotatable knob, a mechanical dial, a calibration screw, an electronic button, an electrical adjuster, a digital touchscreen display, etc., for selectively actuating the intensity of light transmitted through light splitter connector (120). By way of example only, adjustment gauge (150) may comprise a knob that is manually actuated by an operator to selectively adjust the illuminance directed toward guidewire (50) and endoscope (60), respectively. In other versions, adjustment gauge (150) comprises a computer based program displayed on a digital screen such that an operator accesses a digital dial or other digital control to manipulate the illuminance directed to guidewire (50), endoscope (60), and any other instrument included in illuminating system (100). Adjustment gauge (150) may take other suitable forms as will be apparent to those of ordinary skill in the art in view of the teachings herein. Alternatively, adjustment gauge (150) may be omitted in some versions.

Although not shown, illuminating system (100) may further comprise a conventional light detector that includes a sensor. The sensor of the light detector is operable to generate electrical signals based on light received by the sensor. The light detector may further include hardware that is configured to process those generated electrical signals and generate an output that provides feedback to the operator relating to the light received by the light detector. Such feedback may include audible feedback (e.g., an audible tone, a voice providing spoken words, etc.), visual feedback (e.g., a selectively illuminating LED, a graphical interface providing graphic and/or textual feedback, etc.), and/or tactile feedback (e.g., a feature providing a vibration through a handpiece associated with the instruments disclosed above. Various suitable forms that operator feedback may take will be apparent to those of ordinary skill in the art in view of the teachings herein. In some instances, it may be desirable to configure the light detector to be operable to “subtract” any unwanted light from light scattering, reflection, or other optical phenomena so as to improve upon the information indicated by the light detector. Various suitable ways in which such subtraction may be provided will be apparent to those of ordinary skill in the art in view of the teachings herein.

By way of example only, illuminating system (100) may include a light detector in accordance with at least some of the teachings of U.S. Pub. No. 2016/0287083, entitled “Illuminating Guidewire with Optical Sensor,” published Oct. 6, 2016, the disclosure of which is incorporated by reference herein. In the present example, based upon the characteristics of the reflected light (e.g., intensity, color, etc.), the light detector may be operable to indicate a distance between the distal end of guidewire (50) or endoscope (6) and the anatomical structure(s) that surrounds the distal end, as well as the color of such anatomical structure(s). In addition, the light detector, based upon quantitative optical spectroscopy, optical coherence tomography, and/or other optical processing techniques, may indicate a distance between the distal end of guidewire (50) or endo scope (60) and the anatomical structure(s) that surround the distal end, as well as the type and/or pathology of anatomical structure(s) that surrounds the distal end of guidewire (50) or endoscope (60), respectively. By way of example only, as the distal end of guidewire (50) or endoscope (60) is advanced toward a wall of an anatomical structure, the intensity of light reflected toward the distal end increases, thus indicating that the distal end of guidewire (50) or endoscope (60) is approaching an anatomical structure.

In use, an operator connects light source (110) to light splitter connector (120) with cable (130) such that luer connector (132) connects cable (130) to light source (110), and luer connector (134) connects cable (130) to light splitter connector (120) at corresponding luer connector (122). Light splitter connector (120) is further connected to endoscope (60) by attaching light post (70) to another corresponding luer connector (124), such that optical communication is established between endoscope (60) and light source (110). An operator connects cable (140) to light splitter connector (120) by attaching luer connector (142) of cable (140) to corresponding luer connector (126) of light splitter connector (120). In this instance, guidewire (50) is connected to illuminating system (100) by connecting connector (55) to luer connector (144) such that light source (110) is now in optical communication with guidewire (50).

With both guidewire (50) and endoscope (60) included in illuminating system (100), an operator may utilize guidewire (50) and endoscope (60) in a procedure and selectively activate light source (110) when illumination is required from either instrument (50, 60). In other words, with guidewire (50) and endoscope (60) optically connected to light source (110), an operator activates light source (110) to transmit light through cables (130, 140) and light splitter connector (120) to thereby emit illumination through the distal end of guidewire (50) or transparent window (66) of endoscope (60), respectively. With guidewire (50) and/or endoscope (60) receiving light therein, an operator may illuminate the site in the patient distal to the distal end of guidewire (50) or transparent window (66). Providing illumination through the distal end of guidewire (50) or transparent window (66) of endoscope (60) allows an operator to visually confirm the positioning of the distal end of guidewire (50) or endoscope (60) in the anatomical passageway of the patient with relative ease.

An operator selectively actuates adjustment gauge (150) to transition the intensity or illuminance of light transmitted towards either guidewire (50) or endoscope (60), respectively. As a merely illustrative example, an operator may redirect a substantial intensity of the light produced by light source (110) to light post (70) such that the light communicating features of shaft (64) receive a higher intensity light through transparent window (66). In this instance, a lower level of luminance is relayed through light splitter connector (120) and cable (140) to guidewire (50). By way of another example, an operator may selectively actuate adjustment gauge (150) to direct a greater luminous intensity from light source (110) to guidewire (50), rather than endoscope (60). In this instance, light post (70) receives less light from light source (110) such that the distal end of guidewire (50) emits a brighter intensity of light. Adjustment gauge (150) is adjustable to direct various other suitable intensities of illumination to guidewire (50) and endoscope (60), respectively, as will be apparent to those of ordinary skill in the art in view of the teachings herein. For instance, in some versions, adjustment gauge (150) is operable to provide communication of light to only either guidewire (50) or endoscope (60), without the other of guidewire (50) or endoscope (60) receiving any light at that time.

In instances where the light detector is included in illuminating system (100), the light detector is operable to determine and/or indicate the presence and/or characteristics of light reflected by the anatomical structure(s) that surround the distal end of guidewire (50) and/or endoscope (60) back towards the illuminating fibers of the instrument. This reflected light is transmitted through the illuminating fibers and is emitted from the guidewire (50) or endoscope (60) through the light splitter connector (120) and toward the light detector such that the detector is able to determine and/or indicate the presence of anatomical structure(s) that are distal to the distal end of guidewire (50) and/or endoscope (60), respectively. Based on the detected light that is reflected back from anatomical structure(s) that are distal to the distal end of guidewire (50) and/or endoscope (60), the light detector and/or components that are coupled with the detector may further provide real-time feedback to the operator concerning the position of guidewire (50) or endoscope (60) and/or the anatomical structure(s) that are distal to the distal end of either guidewire (50) and endoscope (60).

Although two instruments (50, 60) are shown connected to light source (110), it should be understood that illuminating system (100) may include fewer or additional instruments optically coupled thereto. Although not shown, it will be apparent to those of ordinary skill in the art that light splitter connector (120) may first be connected to connector (55) of guidewire (50), rather than light post (70) of endoscope (60), such that endoscope (60) is connected to light splitter connector (120) via cables (130, 140). In addition, while guidewire (50) and endoscope (60) are incorporated into system (100) in this example, any other suitable kinds of instruments may be incorporated into system (100) as will be apparent to those of ordinary skill in the art in view of the teachings herein. By way of example only, endoscope (60) may be replaced with any other suitable kind of bore scope or other instrument that receives and transmits light.

B. Illuminating System Using Light Source with Integral Light Splitter

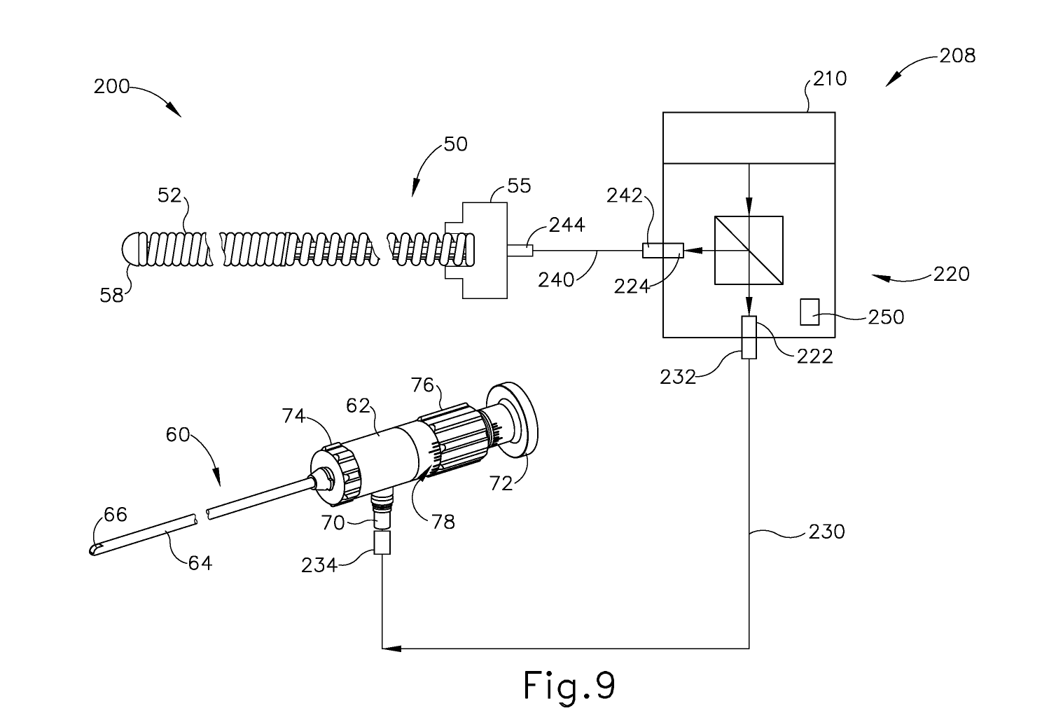

FIG. 9 shows an exemplary alternative illuminating system (200) comprising a light assembly (208) and a pair of light cables (230, 240). Light assembly (208) of the present example comprises an integral light source (210) and an integral light splitter (220). Except as otherwise described below, illuminating system (200), light source (210), light splitter (220), and light cables (230, 240) may be configured and operable just like illuminating system (100), light source (110), light splitter connector (120), and light cables (130, 140), respectively, described above. Similar to illuminating system (100) described above, illuminating system (200) is operable to supply light from light source (210) to multiple instruments connected therein, through light splitter (220) and a series of cables (230, 240), to thereby provide the instruments with sufficient lighting for use in a procedure.

In the present example, light source (210) and light splitter (220) are integrally assembled in light assembly (208) such that a single, unitary piece of capital equipment is configured and operable to provide the light and bifurcate the light to at least two instruments connected thereto. In other examples, light source (210) and light splitter (220) are assembled together to form light assembly (208) such that the joinder of the multiple components into a single assembly effectively becomes configured and operable to provide the light and bifurcate the light at the same stage. It should be understood that illuminating system (200) of the present example may be readily combined with guidewire (50) and endoscope (60) described above.

In the present example, as seen in FIG. 9, guidewire (50) and endoscope (60) are included in illuminating system (200) such that illuminating system (200) provides guidewire (50) and endoscope (60) with sufficient lighting to allow an operator to navigate the instruments (50, 60) within a patient. Although not shown, it should be understood that illuminating system (200) may be connected to other instruments, in addition to or in place of endoscope (60) and guidewire (50), that require a source of illumination. Light splitter (220) is a bifurcation tool configured to receive and distribute light from light source (210) as will be apparent to those of ordinary skill in the art. In the present example, light assembly (208) is configured such that light splitter (220) distributes the light of light source (210) through cables (230, 240), endoscope (60), and guidewire (50). In particular, light assembly (208) couples light splitter (220) and light source (210) such that light splitter (220) is in direct communication with light source (210), rather than being indirectly connected to light source (210) through the connections of at least one cable, such as in illuminating system (100) described above. In this instance, with light splitter (220) directly connected to light source (210), the light emitted from light source (210) is immediately bifurcated by light splitter (220) prior to the light being transmitted through cables (230, 240).

Light cable (230) comprises a pair of luer connectors (232, 234) positioned at opposite ends of light cable (230). It should be understood that luer connectors (232, 234) are configured and operable to be interchangeable with one another. Cable (230) is optically coupled on one end to light splitter (220) by the engagement of luer connector (232) and corresponding luer connector (222) of light splitter (220). Cable (230) is coupled on the opposing end to endoscope (60) by the engagement of luer connector (234) and light post (70). With cable (230) optically connected to both light splitter (220) and endoscope (60), light source (210) establishes optical communication with endoscope (60) through light splitter (220). In other words, light splitter (220) of light assembly (208) is configured to receive luer connector (232), at corresponding luer connector (222), and opposing luer connector (234) of cable (230) receives light post (70) of endoscope (60) such that light splitter (220) is operable to transmit light from light source (210) and through cable (23) toward endoscope (60). With light post (70) in optical communication with the light communicating features in shaft (64) of endoscope (60), light source (210) of light assembly (208) is operable to supply the light communicating features of endoscope (60) with lighting through light splitter (220) and cable (230) such that endoscope (60) becomes operable to direct light through transparent window (66).

Light splitter (220) of light assembly (208) is further configured to receive a second light cable (240) at another corresponding luer connector (224). In particular, second cable (240) includes a pair of luer connectors (242, 244) positioned at opposite ends of cable (240). Similar to luer connectors (232, 234) of cable (230), luer connectors (242, 244) of cable (240) are configured and operable to be interchangeable with one another. It should be understood that cable (240) is configured and operable just like cables (130, 140, 230) such that cables (130, 140, 230, 240) are interchangeable. Additionally, it should be understood that corresponding luer connectors (222, 224) of light splitter (220) are similarly configured and operable such that corresponding luer connectors (222, 224) are interchangeable. In other words, any corresponding luer connector (222, 224) is configured to receive any luer connector (232, 234, 242, 244) of cable (230, 240) or light post (70) of endoscope (60) therein.

Luer connector (242) of cable (240) is coupled to corresponding luer connector (224), while the opposite luer connector (244) of cable (240) is coupled to connector (55) of guidewire (50). In this instance, light source (210) of light assembly (208) is in optical communication with endoscope (60) through the connection between light splitter (220) and cable (230), and in further communication with guidewire (50) through the connection between light splitter (220) and cable (240). Thus, light source (210) is operable to transmit light to guidewire (50) such that illumination fiber (56) receives lighting and transmits the light to lens (58). Although light splitter (220) is shown to include two corresponding luer connectors (222, 224), it should be understood that light splitter (220) may include additional corresponding luer connectors (222, 224) such that light splitter (220) is configured to connect to more cables and/or instruments to light source (210).

Similar to light splitter connector (120), light splitter (220) of the present example includes an adjustment gauge (250) that is configured to selectively adjust the intensity of light reflected from light source (210) and toward guidewire (50) and endoscope (60), respectively. Adjustment gauge (250) is operable to transition the luminous intensity transmitted from light source (210) to each instrument connected within illuminating system (200). In the present example, adjustment gauge (250) has a slotted window (not shown) within light splitter (220) that is selectively adjustable to control the luminance transmitted to the respective instruments connected to light splitter (220). As described above, adjustment gauge (250) may alternatively comprise a series of apertures or holes (not shown) that have varying diameters. In this instance, the intensity of light transmitted to guidewire (50) or endoscope (60) is determined by the particular diameter of the hole selected by an operator. Adjustment gauge (250) may take various suitable forms as described above with respect to adjustment gauge (150) and as will be apparent to those of ordinary skill in the art in view of the teachings herein. As noted above, adjustment gauge (250) is merely optional and may be omitted if desired.

In use, an operator utilizes light assembly (208) by connecting light source (210) to light splitter (220) by attaching light splitter (220) to light source (210). Although not shown, it should be understood that light splitter (220) is integrally attached to light source (210) through an engagement means as will be apparent to those of ordinary skill in the art. Alternatively, in some examples light assembly (208) may be preconstructed such that light source (210) and light splitter (220) are assembled into light assembly (208) as a single, integral unit. In this instance, an operator is not required to attach light source (210) to light splitter (220) as light assembly (208) is preassembled with light source (210) and light splitter (220) already connected. With light splitter (220) optically connected to light source (210), an operator assembles cables (230, 240) to light splitter (220). In particular, luer connector (232) of cable (230) is coupled to corresponding luer connector (222) of light splitter (220), and luer connector (234) is coupled to corresponding luer connector (224). The opposing luer connector (234) of cable (230) is connected to endoscope (60) by coupling light post (70) to luer connector (234), such that optical communication is established between endoscope (60) and light source (210). The opposing luer connector (244) of cable (240) is connected to guidewire (50) by coupling connector (55) to luer connector (244), such that optical communication is established between guidewire (50) and light source (210).

With both guidewire (50) and endoscope (60) included in illuminating system (200), an operator utilizes guidewire (50) and endoscope (60) in a procedure and selectively activates light assembly (208) when illumination is required from either instrument (50, 60). In other words, with guidewire (50) and endoscope (60) optically connected to light assembly (208), an operator activates light source (210) to transmit light through light splitter (220) and cables (230, 240) to thereby emit illumination through the distal end of guidewire (50) or transparent window (66) of endoscope (60), respectively. With guidewire (50) and/or endoscope (60) receiving light therein, an operator illuminates the site in the patient distal to the distal end of guidewire (50) or transparent window (66). Providing illumination through the distal end of guidewire (50) or transparent window (66) of endoscope (60) allows an operator to visually confirm the positioning of the distal end of guidewire (50) or endoscope (60) in the anatomical passageway of the patient with relative ease.

An operator selectively actuates adjustment gauge (250) to transition the intensity or illuminance of light transmitted towards either guidewire (50) or endoscope (60), respectively. Adjustment gauge (250) is adjustable to direct various suitable intensities of illumination to guidewire (50) and endoscope (60), respectively, as will be apparent to those of ordinary skill in the art in view of the teachings herein. Although two instruments (50, 60) are shown connected to light assembly (208), it should be understood that illuminating system (200) may include fewer or additional instruments optically coupled therein.

V. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A guide system comprising: (a) a first instrument, wherein the first instrument comprises at least one light communicating feature, wherein a distal portion of the first instrument is configured to fit within a nasal cavity of a patient, wherein the distal portion of the first instrument is further configured to project light from the at least one light communicating feature of the first instrument; (b) a second instrument, wherein the second instrument comprises at least one light communicating feature, wherein a distal portion of the second instrument is configured to fit within a nasal cavity of a patient, wherein the distal portion of the second instrument is further configured to project light from the at least one light communicating feature of the second instrument; (c) a light source, wherein the light source is operable to simultaneously project light to the first and second instruments through at least one light cable such that the at least one light cable is configured to optically couple the light source to the first and second instruments; and (d) a light splitter, wherein the light splitter is in optical communication with the light source and the first and second instruments such that the light splitter is interposed between the light source and the first and second instruments, wherein the light splitter is configured to selectively bifurcate the light projected from the light source to the first and second instruments.

### Example 2

The guide system of Example 1, wherein the light splitter is connected to the light source and the first and second instruments through the at least one light cable.

### Example 3

The guide system of any one or more of Examples 1 through 2, wherein the light splitter includes an adjustment gauge.

### Example 4

The guide system of Example 3, wherein the adjustment gauge is configured to selectively manipulate the intensity of light directed towards each of the first and second instruments.

### Example 5

The guide system of any one or more of Examples 1 through 4, further comprising a third instrument, wherein the third instrument comprises at least one light communicating feature.

### Example 6

The guide system of Example 5, wherein the light source is operable to simultaneously project light to the first, second, and third instruments through at least one light cable.

### Example 7

The guide system of Example 6, wherein the light splitter is in optical communication with the third instrument such that the light source is interposed between the light source and the third instrument.

### Example 8

The guide system of any one or more of Examples 1 through 7, wherein first instrument comprises a guide member.

### Example 9

The guide system of Example 8, wherein the guide member comprises a guidewire.

### Example 10

The guide system of any one or more of Examples 1 through 9, wherein the at least one light communicating feature of the first instrument comprises at least one illumination fiber.

### Example 11

The guide system of Example 10, wherein the first instrument comprises a guidewire, wherein the at least one illumination fiber is operable to distally transmit the light projected by the light source from a proximal end of the guidewire to the distal end of the guidewire.

### Example 12

The guide system of any one or more of Examples 1 through 11, wherein the first instrument comprises an endoscope.

### Example 13

The guide system of Example 12, wherein the light communicating feature of the second instrument comprises a light pipe disposed within the endoscope

### Example 14

The guide system of any one or more of Examples 1 through 13, wherein the light splitter is integral with the light source such that the light splitter is in direct optical communication with the light source.

### Example 15

The guide system of Examples 1 through 14, wherein the at least one cable comprises two cables such that the first and second instruments are each connected to the light splitter by a respective one of the two cables.

### Example 16

A positioning system comprising: (a) a guide member comprising at least light communicating feature; (b) an endoscope comprising at least one light communicating feature; (c) a light source in optical communication with the guide member and the endoscope such that the light source is operable to project light to the guide member and the endoscope; (d) a light splitter configured to split light projected by the light source and thereby communicate the split light to the guide member and to the endoscope; and (e) at least one light cable configured to connect the guide member, the endoscope, the light source and the light splitter such that the at least one light cable is operable to transmit the light projected from the light source through the light splitter and to the guide member and the endoscope.

### Example 17

The positioning system of Example 16, wherein the light splitter comprises an adjustment gauge configured to selectively adjust the intensity of light directed towards the guide member and endoscope.

### Example 18

The positioning system of any one or more of Examples 16 through 17, wherein the light splitter is integral with the light source such that the light source is in direct communication with the guide member and the endoscope.

### Example 19

The positioning system of any one or more of Examples 16 through 18, wherein the light cable is configured to connect the guide member to the light splitter and a second light cable is configured to connect the endoscope to the light splitter.

### Example 20

A guide system comprising: (a) a first instrument, wherein the first instrument comprises a light communicating feature, wherein a distal portion of the first instrument is configured to fit within a nasal cavity of a patient, wherein the distal portion of the first instrument is further configured to project light from the at least one light communicating feature of the first instrument; (b) a second instrument, wherein the second instrument comprises a light communicating feature, wherein a distal portion of the second instrument is configured to fit within a nasal cavity of a patient, wherein the distal portion of the second instrument is further configured to project light from the at least one light communicating feature of the second instrument; and (c) a light assembly, wherein the light assembly is operable to project light to the first and second instruments through at least one light cable such that the at least one light cable is configured to optically couple the light assembly to the first and second instruments, wherein the light assembly is further operable to selectively split the light projected to each of the first and second instruments such that the light assembly is operable to project different intensities of light to each of the first and second instruments.

VI. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A guide system comprising:
(a) a first instrument, wherein the first instrument comprises at least one light communicating feature, wherein a distal portion of the first instrument is configured to fit within a nasal cavity of a patient, wherein the distal portion of the first instrument is further configured to project light from the at least one light communicating feature of the first instrument;
(b) a second instrument, wherein the second instrument comprises at least one light communicating feature, wherein a distal portion of the second instrument is configured to fit within a nasal cavity of a patient, wherein the distal portion of the second instrument is further configured to project light from the at least one light communicating feature of the second instrument;
(c) a light source, wherein the light source is operable to simultaneously project light to the first and second instruments through at least one light cable such that the at least one light cable is configured to optically couple the light source to the first and second instruments; and
(d) a light splitter, wherein the light splitter is in optical communication with the light source and the first and second instruments such that the light splitter is interposed between the light source and the first and second instruments, wherein the light splitter is configured to selectively bifurcate the light projected from the light source to the first and second instruments.

2. The guide system of claim 1, wherein the light splitter is connected to the light source and the first and second instruments through the at least one light cable.

3. The guide system of claim 1, wherein the light splitter includes an adjustment gauge.

4. The guide system of claim 3, wherein the adjustment gauge is configured to selectively manipulate the intensity of light directed towards each of the first and second instruments.

5. The guide system of claim 1, further comprising a third instrument, wherein the third instrument comprises at least one light communicating feature.

6. The guide system of claim 5, wherein the light source is operable to simultaneously project light to the first, second, and third instruments through at least one light cable.

7. The guide system of claim 6, wherein the light splitter is in optical communication with the third instrument such that the light source is interposed between the light source and the third instrument.

8. The guide system of claim 1, wherein first instrument comprises a guide member.

9. The guide system of claim 8, wherein the guide member comprises a guidewire.

10. The guide system of claim 1, wherein the at least one light communicating feature of the first instrument comprises at least one illumination fiber.

11. The guide system of claim 10, wherein the first instrument comprises a guidewire, wherein the at least one illumination fiber is operable to distally transmit the light projected by the light source from a proximal end of the guidewire to the distal end of the guidewire.

12. The guide system of claim 1, wherein the first instrument comprises an endoscope.

13. The guide system of claim 12, wherein the light communicating feature of the second instrument comprises a light pipe disposed within the endoscope.

14. The guide system of claim 1, wherein the light splitter is integral with the light source such that the light splitter is in direct optical communication with the light source.

15. The guide system of claim 14, wherein the at least one cable comprises two cables such that the first and second instruments are each connected to the light splitter by a respective one of the two cables.

16. A positioning system comprising:
(a) a guide member comprising at least light communicating feature;
(b) an endoscope comprising at least one light communicating feature;
(c) a light source in optical communication with the guide member and the endoscope such that the light source is operable to project light to the guide member and the endoscope;
(d) a light splitter configured to split light projected by the light source and thereby communicate the split light to the guide member and to the endoscope; and
(e) at least one light cable configured to connect the guide member, the endoscope, the light source and the light splitter such that the at least one light cable is operable to transmit the light projected from the light source through the light splitter and to the guide member and the endoscope.

17. The positioning system of claim 16, wherein the light splitter comprises an adjustment gauge configured to selectively adjust the intensity of light directed towards the guide member and endoscope.

18. The positioning system of claim 16, wherein the light splitter is integral with the light source such that the light source is in direct communication with the guide member and the endoscope.

19. The positioning system of claim 18, wherein the light cable is configured to connect the guide member to the light splitter and a second light cable is configured to connect the endoscope to the light splitter.

20. A guide system comprising:
(a) a first instrument, wherein the first instrument comprises a light communicating feature, wherein a distal portion of the first instrument is configured to fit within a nasal cavity of a patient, wherein the distal portion of the first instrument is further configured to project light from the at least one light communicating feature of the first instrument;
(b) a second instrument, wherein the second instrument comprises a light communicating feature, wherein a distal portion of the second instrument is configured to fit within a nasal cavity of a patient, wherein the distal portion of the second instrument is further configured to project light from the at least one light communicating feature of the second instrument; and
(c) a light assembly, wherein the light assembly is operable to project light to the first and second instruments through at least one light cable such that the at least one light cable is configured to optically couple the light assembly to the first and second instruments, wherein the light assembly is further operable to selectively split the light projected to each of the first and second instruments such that the light assembly is operable to project different intensities of light to each of the first and second instruments.
